(19) United States
(12) Patent Application Publication
(10) Pub. No.: US 2013/0316933 A1
(43) Pub. Date: Nov. 28, 2013

(54) METHOD FOR THE DIAGNOSIS, PROGNOSIS AND MONITORING OF MUSCULAR DEGENERATION

(71) Applicants: Rosario Osta Pinzolas, Zaragoza (ES); Maria Jesus Munoz Gonzalvo, Zaragoza (ES); Pilar Zaragoza Fernandez, Zaragoza (ES); Ana Cristina Calvo Royo, Zaragoza (ES); Raquel Manzano Martinez, Zaragoza (ES); Alberto Garcia Redondo, Zaragoza (ES); Paz Torre Merino, Zaragoza (ES)

(72) Inventors: Rosario Osta Pinzolas, Zaragoza (ES); Maria Jesus Munoz Gonzalvo, Zaragoza (ES); Pilar Zaragoza Fernandez, Zaragoza (ES); Ana Cristina Calvo Royo, Zaragoza (ES); Raquel Manzano Martinez, Zaragoza (ES); Alberto Garcia Redondo, Zaragoza (ES); Paz Torre Merino, Zaragoza (ES)

(73) Assignee: Universidad de Zaragoza, Zaragoza (ES)

(21) Appl. No.: 13/992,580

(22) PCT Filed: Dec. 7, 2011
(86) PCT No.: PCT/ES11/00351

§ 371 Date: Aug. 15, 2013

(30) Foreign Application Priority Data
Dec. 9, 2010 (ES) P201031814

(51) Int. Cl. C12Q 1/68

(52) U.S. Cl. CPC C12Q 1/6883; G06F 19/345
USPC 506/9; 435/6.11; 435/7.1; 530/389.1; 536/24.31; 536/24.33; 506/16; 702/20

(57) ABSTRACT

The invention relates to methods based on the quantification of a set of biomarkers, preferably in biological samples isolated from skeletal muscle, for performing the diagnosis, prognosis and/or monitoring of muscular degeneration, preferably muscular degeneration caused by motor neuron diseases, more preferably amyotrophic lateral sclerosis (ALS); and to a kit for the diagnosis, prognosis and monitoring of said type of diseases. The method in the invention for the prognosis and/or monitoring of muscular degeneration makes it possible to determine the rate of progression of said degeneration (fast or slow rate of progression in relation to the normal rate of progression).

The present invention is in the field of molecular biology and medicine, specifically in the methods based on quantification of expression of biomarkers for diagnosis, prognosis and/or monitoring muscular degeneration, preferably muscular degeneration caused by motor neurone diseases, more preferably muscular degeneration caused by amyotrophic lateral sclerosis (ALS) as well as in kits for diagnosis, prognosis and/or monitoring these types of diseases.

### STATE OF PRIOR ART

Amyotrophic lateral sclerosis (ALS), also known as Lou Gehrig's disease, is a neurodegenerative disease that causes a progressive degeneration of the motor neurones that control voluntary muscles, leading to their irreversible loss and consequently the patient's death. This disease belongs to the group of conditions called diseases of the motor neurones or motor neurone diseases.

ALS is one of the most common motor neurone diseases in the world and affects people of all races and ethnicities. It is the third most common cause of death from neurodegenerative disease in adults, after Alzheimer's and Parkinson's. It usually affects people between 40 and 60 years of age, although it can develop in younger or older people and is more frequent in men than women.

Motor neurones are nerve cells located in the brain, brain stem and spinal cord that serve as control units and vital communication links between the nervous system and the voluntary muscles of the body. Messages from cerebral motor neurones (called the upper motor neurones) are transmitted to the motor neurones in the spinal cord (lower motor neurones) and from there to each particular muscle. In ALS, both upper motor neurones and lower motor neurones degenerate or die and stop sending messages to the muscles, which functionally impaired, gradually weaken, atrophy and contract (twitching) finally leading to paralysis. Therefore, ALS causes weakness that is manifested in a wide range of disabilities, which eventually affect all the muscles that are under voluntary control causing them to lose their ability to control movement. In addition, when the muscles of the diaphragm and the chest wall fail, patients lose the ability to breathe without a ventilator or artificial respirator. The majority of people with ALS die from respiratory failure, generally between 3 to 5 years after the start of the symptoms. However, around 10% of patients with ALS survive 10 years or more.

Currently, the aetiology of ALS is unknown, although various possible causes of the neurodegeneration have been proposed such as excitotoxicity, excessive excitatory tone, denatured proteins, defective energy production, abnormal calcium metabolism and transport and activation of proteases and endonucleases. Out of all cases of ALS, some 90% to 95% occur apparently randomly (sporadic ALS) without any clearly associated risk factor. Under these circumstances, patients do not have a family history of the disease and members of their family are not considered to have a higher risk of developing it. By contrast, the familial form of ALS, occurring in 5% to 10% of all cases, generally results from a hereditary pattern characterised as autosomal dominant inheritance, although cases of familial ALS have been described that are attributed to autosomal recessive inheritance. Some 20% of all familial cases result from a specific mutation in the enzyme known as superoxide dismutase 1 (SOD1). However, not all familial cases of ALS are due to this mutation, therefore it is clear that there are other unidentified genetic causes.

As regards the diagnosis of the disease, there are various clinical tests used routinely (Merit Cudkowicz, et al., 2004, NeuroRx: The Journal of the American Society for Experimental NeuroTherapeutics, 1:273-283), particularly electromyography (EMG). However, the delay between the onset of the symptoms to the definitive clinical diagnosis can often take several months, limiting the use of an effective therapy. This time lapse could be dramatically reduced by the use of biomarkers. Therefore, biomedical research studies of this disease have mainly focussed on the search for diagnosis and prognosis biomarkers that are able to provide the necessary clinical information for applying more effective treatment, which could even take place before the onset of symptoms. These biomarkers would also enable monitoring the efficacy of drugs administered during treatment or investigated during clinical trials. Biological fluids and tissues that have been used for detecting these biomarkers in animal models and ALS patients have mainly been serum and cerebrospinal fluid, spinal cord and brain (Ryberg H., Bowser R., 2008, Proteomics, 5:249-262; Ryberg H. et al., 2010, Muscle & Nerve, 42:104-111). In this sense, biomarkers for the diagnosis and detection of the progression of ALS from samples of blood, plasma, serum or cerebrospinal fluid have been proposed (WO2010061283; WO2008044213).

Skeletal muscle is crucial in clinical diagnosis from the point of view of EMG. Furthermore, taking into account that this tissue is one of those most damaged by the disease and EMG can be carried out in a less invasive way than by taking cerebrospinal fluid and in an earlier stages of the disease, this has also been investigated in some genetic expression analyses in transgenic mouse models of the disease such as mice expressing human SOD1 with mutations in positions G86R or G93A (Gonzalez de Aguilar, J. L. et al., 2008, Physiological Genomics, 32:207-218; Kevin H. J. Park and Inez Vincent, 2008, Biochim Biophys Acta, 1782(7-8):462-468).

However, despite the efforts made to date, there is no reliable biomarker that can be used in clinical practice for the diagnosis or prognosis of ALS, so such a discovery still remains necessary. The identification of such molecular biomarkers would enable early diagnosis of the disease, which would in turn enable early administration of an effective treatment. Furthermore, this would provide a valuable tool that would help monitor the effects of therapies administered to the patient and follow the progress or development of the disease.

### DESCRIPTION OF THE INVENTION

The present invention provides methods based on quantification of a set of biomarkers for carrying out the diagnosis, prognosis and/or monitoring of muscular degeneration, preferably muscular degeneration caused by motor neurone diseases, more preferably of the muscular degeneration caused by amyotrophic lateral sclerosis (ALS) and also a kit for the diagnosis, prognosis and/or monitoring of these types of diseases.

Because the biomarkers quantified in the methods of the present invention are preferably measured in isolated biological samples of skeletal muscle, the main tissue affected by muscular degeneration in ALS, the expression pattern of these biomarkers in this tissue damaged by the disease is representative of situations of muscular degeneration. This muscular degeneration is a process common to various diseases affecting skeletal muscle. Therefore, the methods of the invention are useful for the diagnosis, prognosis and/or monitoring of muscular degeneration caused by both myopathic diseases and by neuromuscular diseases, although they are preferably useful in the diagnosis, prognosis and/or monitoring of muscular degeneration caused by motor neurone diseases such as, for example but without limitation, ALS.

In the present invention, it is demonstrated that the Col19α1 gene is overexpressed in isolated biological samples of patients with muscular degeneration such as, for example but without limitation, ALS patients compared to healthy individuals without this degeneration. Therefore this gene can be considered to be a biomarker applicable in clinical practice for the early diagnosis of muscular degeneration, with the advantage compared to other routine detection methods for these types of degenerative processes that it enables reducing the delay between onset of symptoms and establishing a diagnosis, which enables the administration of a treatment from the early stages of the disease that causes this degeneration.

Therefore, one aspect of the invention refers to the use of the Col19α1 gene or of its expression products for the diagnosis, prognosis and monitoring of muscular degeneration. In a preferred embodiment of this aspect of the invention, muscular degeneration is caused by a motor neurone disease. In a more preferred embodiment, the motor neurone disease is selected from the list comprising: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy or amyotrophic lateral sclerosis. In a still more preferred embodiment, the motor neurone disease is amyotrophic lateral sclerosis.

The Col19α1 or Col19a1 gene is the “collagen, type XIX, alpha 1” gene, GenBank reference number (Gen ID) 12823 in mouse and 1310 in human, and its functions have been related to cellular adhesion, organisation of the extracellular matrix and cellular development and differentiation of skeletal muscle such as, for example, the oesophageal muscle, and others.

In addition to the Col19α1 gene, another gene, IMPA1, is overexpressed in isolated biological samples, particularly lymphocytes, of patients with muscular degeneration such as, for example but without limitation, the case of ALS patients compared to healthy individuals without this degeneration. Therefore, this gene can also be considered as a biomarker applicable in clinical practice for the early diagnosis of muscular degeneration. Thus, another aspect of the invention refers to the use of the genes Col19α1 and/or IMPA1 or of their expression products for the diagnosis of muscular degeneration. A preferred embodiment of this aspect of the invention refers to the use of the Col19α1 and IMPA1 genes or of their expression products for the diagnosis of muscular degeneration. In another preferred embodiment of this aspect of the invention, the muscular degeneration is caused by a motor neurone disease. In a more preferred embodiment, the motor neurone disease is selected from the list comprising: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy or amyotrophic lateral sclerosis. In a still more preferred embodiment, the motor neurone disease is amyotrophic lateral sclerosis.

The IMPA1 gene is also known as the “inositol (myo)-1(or 4)-monophosphatase 1” gene, GenBank reference number (Gen ID) 55980 in mouse and 3612 in human, and its function is related to homeostasis of inositol.

The present invention demonstrates that the change in the levels of expression of the Col19α1 gene during the muscular degeneration process is significantly correlated with the development of this process; so measurement of this change in gene expression during the degenerative process in isolated biological samples of the patient taken at different times enables determining the speed of progression of muscular degeneration by comparing the values of change of gene expression obtained for the patient with reference levels of change of gene expression. The sample applies to the NOGO A gene. Therefore, these two genes are proposed as biomarkers for the prognosis and monitoring of muscular degeneration. This prognosis and monitoring is useful, for example, for determining the effectiveness of a treatment being administered to a patient and classifying whether the treatment is effective or not effective in that patient.

Thus, another aspect of the invention refers to the use of the Col19α1 and/or NOGO A genes or of their expression products for the prognosis and monitoring of muscular degeneration. A preferred embodiment of this aspect of the invention refers to the use of the Col19α1 and NOGO A genes or of their expression products for the prognosis and monitoring of muscular degeneration. In another preferred embodiment of this aspect of the invention, the muscular degeneration is caused by a motor neurone disease. In a more preferred embodiment, the motor neurone disease is selected from the list comprising: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy or amyotrophic lateral sclerosis. In a still more preferred embodiment, the motor neurone disease is amyotrophic lateral sclerosis.

The NOGO A gene is also known as the reticulon 4 or RTN4 gene, GenBank reference number (Gen ID) 68585 in mouse and 57142 in human, and its function has been related to angiogenesis, apoptosis, negative regulation of axonal regeneration, etc. This gene induces instability in the neuromuscular junction when overexpressed in muscle.

In the case of female individuals, the examples of the present invention demonstrate that, in addition to the Col19α1 and NOGO A genes, changes in the expression levels of a further 6 genes during the process of muscular degeneration is significantly correlated to the development of this process, so that the measurement of this change in gene expression during the degenerative process in various isolated biological samples of the patient at different times enables the determination of the speed of progression of muscular degeneration when comparing values of change of gene expression obtained for each of these genes in the patient with reference levels of change of gene expression given for each gene. Therefore, another aspect of the invention refers to the use of the Col19α1, NOGO A, ANKRD1, SNX10, MYOG, MYOD1, NNT and/or SLN genes or of their expression products for the prognosis and monitoring of muscular degeneration in a female individual. A preferred embodiment of this aspect of the invention refers to the use of the Col19α1, NOGO A, ANKRD1, SNX10, MYOG, MYOD1, NNT and SLN genes or of their expression products for the prognosis and monitoring of muscular degeneration in a female individual. In another preferred embodiment of this aspect of the invention, the muscular degeneration is caused by a motor neurone disease. In a more preferred embodiment, the motor neurone disease is selected from the list comprising: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy or amyotrophic lateral sclerosis. In a still more preferred embodiment, the motor neurone disease is amyotrophic lateral sclerosis.

The ANKRD1 gene is also known as the “ankyrin repeat domain 1 (cardiac muscle)” gene, GenBank reference number (Gen ID) 107765 in mouse and 27063 in human, and its function has been related to muscle plasticity.

The SNX10 gene is also known as the “sorting nexin 10” gene, GenBank reference number (Gen ID) 71982 in mouse and 29887 in human, and its function has been related to regulation of homeostasis of the endosome.

The MYOG gene is also known as the “myogenin” or “myogenic factor 4” gene, GenBank reference number (Gen ID) 17928 in mouse and 4656 in human, and its function has been related to differentiation of muscle cells.

The MYOD1 gene is also known as the “myogenic differentiation 1” gene, GenBank reference number (Gen ID) 17927 in mouse and 4654 in human, and its functions has been related to myogenesis and muscular differentiation.

The NNT gene is also known as the “nicotinamide nucleotide transhydrogenase” gene, GenBank reference number 18115 in mouse and 23530 in human, and its function has been related to homeostasis of glucose.

The SLN gene is also known as the “sarcolipin” gene, GenBank reference number (Gen ID) 66402 in mouse and 6588 in human, and its function has been related to regulation of calcium transport and muscle contraction-relaxation cycles.

The term “expression product” as used in this description refers to any product of transcription or translation (RNA or protein) of the genes Col19α1, IMPA1, NOGO A, ANKRD1, SNX10, MYOG, MYOD1, NNT or SLN, or of any form resulting from the processing of these transcription or translation products.

The term “diagnosis” is understood to mean the process by which the presence or absence of muscular degeneration is identified, preferably muscular degeneration caused by a motor neurone disease, more preferably muscular degeneration caused by amyotrophic lateral sclerosis. The term “prognosis” refers to the process by which the events that could occur in the development or course of a muscular degeneration process may be predicted, preferably muscular degeneration caused by a motor neurone disease, more preferably muscular degeneration caused by amyotrophic lateral sclerosis. In the context of the present invention, the term “prognosis” refers to the process by which the speed of progression of muscular degeneration is established.

In the present invention, the term “muscular degeneration” is understood as the condition that causes progressive weakness and degeneration of the muscles controlling movement, changing the mobility or functionality of skeletal muscle. Muscular degeneration can be a symptom of a disease included in the myopathies, with the term “myopathies” being understood as any type of inflammatory, distal, myotonic, congenital, mitochondrial, metabolic, primary periodic paralysis or muscular dystrophy myopathy; or it can be a symptom of a neuromuscular disease, which affect the nerves controlling voluntary muscles such as, for example but without limitation, multiple sclerosis or myasthenia gravis; more preferably the neuromuscular disease is a motor neurone disease. A “motor neurone disease” is understood as a degenerative pathology, progressive and fatal, that affects the first motor neurone (upper motor neurone), the second motor neurone (lower motor neurone) or both, and can be sporadic or hereditary. Various types of motor neurone diseases can be differentiated by the function of the type of motor neurone affected and the degree to which it is affected such as, for example but without limitation, primary lateral sclerosis, progressive muscular atrophy, amyotrophic lateral sclerosis (ALS), spinal muscular atrophy (SMA), included within them are SMA type 1 or Werdnig-Hoffman disease, SMA type 2, SMA type 3 or Kugelberg-Welander disease and SMA type 4; bulbar palsy, pseudobulbar palsy, ALS with frontotemporal dementia, benign focal amyotrophy, bulbo-spinal atrophy or Kennedy syndrome, hereditary spastic paraplegia, tropical spastic paraplegia, motor neurone disease associated with lymphoproliferative disease or to paraneoplastic syndrome, multifocal motor pane, spinocerebellar ataxia type 2 and 3, adrenomyeloneuropathy, Allgrove syndrome, post-irradiation motor neuropathy, acute poliomyelitis, post-polio syndrome, lathyrism, konzo or Guam amyotrophic lateral sclerosis.

“Amyotrophic lateral sclerosis” or “ALS” is the neuromuscular degenerative disorder, of sporadic or familial origin, in which the primary and secondary motor neurones gradually reduce their functionality and later die, causing progressive muscular paralysis with a fatal prognosis.

Another aspect of the invention refers to a method for “in vitro” diagnosis of muscular degeneration in an individual, hereinafter called “first method of the invention”, comprising:
a. determining the amount of expression product of the Col19α1 gene in an isolated biological sample of an individual, and
b. comparing the amount determined in step (a) with a reference amount.

The term “isolated biological sample” as used in the first method of the invention refers, but is not limited, to tissues and/or biological fluids taken from an individual, obtained by any method known to a person skilled in the art that serves for that purpose. The biological sample can be a tissue, for example but without limitation, a muscle or skeletal muscle biopsy, or can be a biological fluid, for example but without limitation, blood, plasma, serum or lymph. In a preferred embodiment, the isolated biological sample of the first method of the invention is a lymphocyte or skeletal muscle. The term “lymphocyte” in the present invention is understood as a lymphocyte or a population of lymphocytes and can be obtained by isolation from, for example but without limitation, a blood sample. The skeletal muscle sample can be obtained, for example but without limitation, by extraction from a muscle biopsy of biceps brachii or gluteus superficialis. This sample can be taken from a human, but also from non-human mammals such as, for example but without limitation, rodents, ruminants, felinae or canidae. Therefore, in another preferred embodiment of this aspect of the invention, the individual from which the isolated biological sample comes for the first method of the invention is a mammal. In a more preferred embodiment, the mammal is a human.

The term “reference amount” as used in step (b) of the first method of the invention refers to any value or range of values derived from the quantification of the expression product of the Col19α1 gene in a control biological sample coming from an individual that does not exhibit muscular degeneration. Thus, in another preferred embodiment, the reference amount of step (b) of the first method of the invention is the amount of expression product of the Col19α1 gene in an isolated biological sample of an individual who does not have muscular degeneration.

In another preferred embodiment, the first method of the invention further comprises:
c. assigning the individual of step (a) to the group of patients with muscular degeneration when the amount determined in step (a) is significantly higher than the reference amount.

In another preferred embodiment, the isolated biological sample of the first method of the invention is a lymphocyte, and this method further comprises:
d. determining the amount of expression product of the IMPA1 gene in the lymphocyte of the individual of step (a), and
e. comparing the amount determined in step (d) with a reference amount.

In a more preferred embodiment, the first method of the invention further comprises:
f. assigning the individual of step (a) to the group of patients with muscular degeneration when the amount determined in steps (a) and (d) are significantly higher than the reference amount.

The term “reference amount” as used in step (e) of the first method of the invention refers to any value or range of values derived from the quantification of the expression product of the IMPA1 gene in a control biological sample coming from an individual that does not exhibit muscular degeneration. Thus, in a still more preferred embodiment, the reference amount of step (e) of the first method of the invention is the amount of expression product of the IMPA1 gene in a lymphocyte of an individual that does not exhibit muscular degeneration.

The determination of the amount of expression product of the Col19α1 gene or the IMPA1 gene in an isolated biological sample refers to the measurement of the amount or the concentration, preferably semi-quantitatively or quantitatively. This measurement can be carried out directly or indirectly. Direct measurement refers to the measurement of the amount or the concentration of the gene expression product based on a signal obtained directly from the gene expression product and is directly correlated with the number of molecules of the gene expression product in the sample. This signal, which can also be referred to as the signal intensity, can be obtained, for example, by measuring an intensity value of a chemical or physical property of the expression product. The indirect measure includes the measure obtained of a secondary component (for example a different component from that of gene expression) or a system of biological measurement (for example measurement of cell responses, ligands, labels or products of enzyme reactions).

In accordance with the present invention, determination of the amount of gene expression product can be carried out by any method for determining the amount of gene expression products known by a person skilled in the art. In another preferred embodiment, determination of the amount of gene expression product is carried out by determining the level of mRNA derived from its transcription, after extracting the total RNA from the isolated biological sample, which can be carried out by methods known to a person skilled in the art. The measurement of the mRNA level can be carried out, by way of illustration and without limiting the scope of the invention, by polymerase chain reaction (PCR) amplification, retrotranscription in combination with the ligase chain reaction (RTLCR), retrotranscription in combination with the quantitative polymerase chain reaction (qRT-PCR) or any other method of amplification of nucleic acids; DNA microarrays made with oligonucleotides deposited by any mechanism; DNA microarrays made with oligonucleotides synthesised in situ by photolithography or by any other mechanism; in situ hybridisation using specific probes labelled by any labelling method; by electrophoresis gels; by transfer to a membrane and hybridisation with a specific probe; by NMR or any other image diagnostic technique using paramagnetic nanoparticles or any other type of detectable nanoparticles functionalised with antibodies or by any other means. In another preferred embodiment, determination of the amount of gene expression product is carried out by determining the level of Col19α1 or IMPA1 protein by, for example but without limitation, ELISA, immunohistochemistry or Western blot.

A “significantly higher” amount than a reference amount can be established by a person skilled in the art by the use of different statistical tools, for example but without limitation, by the determination of confidence intervals, determination of the p value, Student's t-test or Fisher's discriminant functions.

Another aspect of the invention refers to a method for the prognosis and “in vitro” monitoring of muscular degeneration in an individual, hereinafter the “second method of the invention” comprising:
a. determining the ΔCt value of the Col19α1 gene in an isolated biological sample of skeletal muscle of an individual that exhibits muscular degeneration,
b. determining the value of ΔCt of the Col19α1 gene in a second isolated biological sample of skeletal muscle of an individual of step (a) obtained at least 1 month after obtaining the isolated biological sample of step (a),
c. calculating the slope of a line obtained after the connecting of the values determined in steps (a) and (b), and
d. comparing the slope calculated in step (c) to a reference value.

In a preferred embodiment, the reference value of step (d) of the second method of the invention is the slope of the line obtained after connecting the mean of the ΔCt values of the Col19α1 gene in various isolated biological samples of skeletal muscle of various individuals exhibiting muscular degeneration to the mean of the ΔCt values of the Col19α1 gene in various biological samples from skeletal muscle of various individuals exhibiting muscular degeneration obtained at least 1 month after obtaining the first biological sample. When the individual of step (a) of the second method of the invention is a male, the isolated biological samples used for the calculation of the reference value of step (d) preferably come from male individuals and when the individual of step (a) of the second method of the invention is a female, the isolated biological samples used for the calculation of the reference value of step (d) preferably come from female individuals.

In a more preferred embodiment, the second method of the invention further comprises:
e. assigning the individual of step (a) to the group of patients with high speed of progression of muscular degeneration when the slope calculated in step (c) is significantly less than the reference value.

In another preferred embodiment, the second method of the invention further comprises:
f. determining the ΔCt value of the NOGO A gene in the isolated biological sample of step (a),
g. determining the ΔCt value of the NOGO A gene in the isolated biological sample of step (b),
h. calculating the slope of a line obtained after connecting the values determined in steps (f) and (g), and
i. comparing the slope calculated in step (h) to a reference value.

In a more preferred embodiment, the reference value of step (i) of the second method of the invention is the slope of a line obtained after connecting the mean of the ΔCt values of the NOGO A gene in various isolated biological samples of skeletal muscle of various individuals exhibiting muscular degeneration to the mean of the ΔCt values of the NOGO A gene in various isolated biological samples of skeletal muscle of various individuals exhibiting muscular degeneration obtained at least 1 month after obtaining the first biological sample. When the individual of step (a) of the second method of the invention is a male, the isolated biological samples used for the calculation of the reference value of step (i) preferably come from male individuals and when the individual of step (a) of the second method of the invention is a female, the isolated biological samples used for the calculation of the reference value of step (i) preferably come from female individuals.

In a still more preferred embodiment, the second method of the invention further comprises:
j. assigning the individual of step (a) to the group of patients with high speed of progression of muscular degeneration when the slopes calculated in steps (c) and (h) are significantly lower than the reference values.

In another preferred embodiment, the individual of the second method of the invention is a female and this method further comprises:
k. determining the ΔCt value of at least one gene selected from the list comprising: ANKRD1, SNX10, MYOG, MYOD1, NNT and SLN in the isolated biological sample of step (a),
l. determining the ΔCt value of the gene(s) selected in step (k) in the isolated biological sample of step (b),
m. calculating the slope(s) of the line(s) obtained for each gene after connecting the values determined in steps (k) and (l), and
n. comparing the slope(s) calculated in step (m) to a reference value.

In a more preferred embodiment, the second method of the invention further comprises:
o. assigning the individual of step (a) to the group of patients with high speed of progression of muscular degeneration when the slopes calculated in steps (c), (h) and (m) are significantly lower than the reference value.

In a still more preferred embodiment, the reference value of step (n) of the second method of the invention is the slope of a line obtained after connecting the mean of the ΔCt values of the ANKRD1, SNX10, MYOG, MYOD1, NNT or SLN genes in various isolated biological samples of skeletal muscle of various female individuals exhibiting muscular degeneration to the mean of the ΔCt values of the ANKRD1, SNX10, MYOG, MYOD1, NNT or SLN genes in various isolated biological samples of skeletal muscle of various female individuals exhibiting muscular degeneration obtained at least 1 month after obtaining the first biological sample.

In order to determine the ΔCt value of a gene in a biological sample, amplification of its expression product must be carried out from the sample, for example but without limitation, by PCR, RTLCR, RT-PCR or qRT-PCR. The term “ΔCt” refers to the normalised threshold value (that is, at the moment of the PCR, RTLCR, RT-PCR or qRT-PCR cycle used for amplification of the gene expression product in which the amplified product starts to appear). For normalisation, amplification of the expression products of one or several control or “housekeeping” genes can be carried out, the level of expression of which is constant over the muscular degeneration process such as, for example but without limitation, the 18S rRNA, GAPDH or β-actin, or any of their combinations.

The isolated biological sample of step (a) of the second method of the invention could be obtained, for example but without limitation, when the individual exhibiting muscular degeneration is diagnosed or even before being administered a treatment or at the time the treatment is administered. The isolated biological sample of step (b) of the second method of the invention could be obtained, as a minimum, one month after obtaining the isolated biological sample of step (a) or at any time after this: preferably at 2 months, 3 months, 4 months, 5 months, 6 months or 7 months after obtaining the isolated biological sample of step (a).

The sample of skeletal muscle of the second method of the invention can be obtained, for example but without limitation, by extracting a muscle biopsy from the biceps brachii or gluteus superficialis. This sample can be taken from a human, but also from non-human mammals such as, for example but without limitation, rodents, ruminants, felinae or canidae. Therefore, in another preferred embodiment of this aspect of the invention, the individual from which the isolated biological sample comes for the second method of the invention is a mammal. In a more preferred embodiment, the mammal is a human.

The values determined in steps (a) and (b), for the Col19α1 gene, (f) and (g) for the NOGO A gene and (k) and (l) for at least one of the ANKRD1, SNX10, MYOG, MYOD1, NNT or SLN genes of the second method of the invention can be used for drawing a line corresponding to each gene, the slope of which can be calculated. This line would represent the ΔCt value in each sample against the time in which the isolated biological samples of steps (a) and (b) were obtained. The calculation of the slope of this line can be carried out by mathematical operations known to a person skilled in the art, with the term “slope” being understood as the value of inclination of this line compared to the horizontal.

The reference values of steps (d), (i) and (n) of the second method of the invention are preferably the slopes of the lines that represent how the expression of the Col19α1, NOGO A, ANKRD1, SNX10, MYOG, MYOD1, NNT and SLN genes change over time in a muscular degeneration process where the speed of progression is normal. Because the level of ΔCt of all these genes reduces progressively with the muscular degeneration process, the slopes of the reference values are negative. Thus, when the slope calculated in any, although preferably in all, of the steps (c), (h) and/or (m) of the second method of the invention are less than the reference value for the gene under study, the individual of step (a) exhibits a high speed of progression of muscular degeneration. In the present invention, the term “high speed of progression” is understood to be the speed of progression of the muscular degeneration process that is above the normal speed of progression in a muscular degeneration process. The normal speed of progression of the muscular degeneration process is determined by the calculated reference values as previously explained.

A “significantly” lower value than the reference value can be established by a person skilled in the art by the use of various statistical tools, for example but without limitation, by determining the confidence intervals, determining the p value, Student's t-test or Fisher's discriminant functions.

In another preferred embodiment, muscular degeneration of an individual of the first or second method of the invention is caused by a motor neurone disease. In a more preferred embodiment, the motor neurone disease is selected from the list comprising: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor pane, benign focal amyotrophy or amyotrophic lateral sclerosis. In a still more preferred embodiment, the motor neurone disease is amyotrophic lateral sclerosis.

Steps (a), (b), (d) and/or (e) of the first method of the invention and steps (a), (b), (c), (d), (f), (g), (h), (i), (k), (l), (m) and/or (n) of the second method of the invention can be partially or totally automated, for example but without limitation, by robotic equipment for the determination of the amount of expression product in step (a) and/or (d) of the first method of the invention or for the determination of the ΔCt value in steps (a), (b), (f), (g), (k) and/or (l) of the second method of the invention.

In addition to the steps described above, the first and second method of the invention can comprise other additional steps, for example but without limitation, related to pre-treatment of the isolated biological samples prior to their analysis or by obtaining a third isolated biological sample of skeletal muscle and its corresponding analysis in the second method of the invention.

Another aspect of the invention refers to a kit, hereinafter the “kit of the invention” that comprises specific primers, probes or antibodies for the Col19α1 gene, or any of their combinations. In a preferred embodiment, the kit of the invention further comprises specific primers, probes or antibodies, or any of their combination, for the NOGO A gene and/or for the IMPA1 gene. In a more preferred embodiment, the kit of the invention further comprises specific primers, probes or antibodies, or any of their combinations, for at least one of the genes selected from the list comprising: ANKRD1, SNX10, MYOG, MYOD1, NNT and SLN.

The primers, probes and/or antibodies included in the kit of the invention are complementary and, therefore, have the ability to hybridise with at least one expression product of the Col19α1, IMPA1, NOGO A, ANKRD1, SNX10, MYOG, MYOD1, NNT and/or SLN genes. In general, the kit of the invention comprises all the necessary reagents to carry out the first and second method of the invention described above. The kit may also include, without any limitation, buffers, enzymes such as, for example but without limitation, polymerases, cofactors to obtain optimal activity from these, agents for preventing contamination, etc. The kit may also include all the necessary supports and recipients for its implementation and optimisation. The kit may also contain other molecules, primers, antibodies, genes, proteins or probes of interest, that may serve as positive or negative controls or for normalising the values obtained. The kit may also preferably contain instructions for carrying out the first and second methods of the invention.

Another aspect of the invention refers to the use of the kit of the invention for diagnosis, prognosis and monitoring of muscular degeneration in an individual. In a preferred embodiment of this aspect of the invention, muscular degeneration is caused by a motor neurone disease. In a more preferred embodiment, the motor neurone disease is selected from the list comprising: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy or amyotrophic lateral sclerosis. In a still more preferred embodiment, the motor neurone disease is amyotrophic lateral sclerosis.

Throughout the description and the claims, the term “comprise” and its variants does not intend to exclude other technical characteristics, additives, components or steps. For people skilled in the art, other aims, advantages and characteristics of the invention will be deduced, partly from the description and partly from the practice of the invention. The following examples and figures are provided for the purposes of illustration and are not intended to be limitations of the present invention.

### DESCRIPTION OF THE FIGURES

FIG. 1. Line showing the change in expression of the Col19α1 gene during the muscular degeneration process in skeletal muscle of the SOD1^{G93A} male mouse model of ALS.

FIG. 2. Line showing the change in expression of the NOGO A gene during the muscular degeneration process in skeletal muscle of the SOD1^{G93A} male mouse model of ALS.

FIG. 3. Line showing the change in expression of the Col19α1 gene during the muscular degeneration process in skeletal muscle of the SOD1^{G93A} female mouse model of ALS.

FIG. 4. Line showing the change in expression of the NOGO A gene during the muscular degeneration process in skeletal muscle of the SOD1^{G93A} female mice model of ALS.

FIG. 5. Line showing the change in expression of the ANKRD1 gene during the muscular degeneration process in skeletal muscle of the SOD1^{G93A} female mice model of ALS.

FIG. 6. Line showing the change in expression of the SNX10 gene during the muscular degeneration process in skeletal muscle of the SOD1^{G93A} female mice model of ALS.

FIG. 7. Line showing the change in expression of the MYOG gene during the muscular degeneration process in skeletal muscle of the SOD1^{G93A} female mice model of ALS.

FIG. 8. Line showing the change in expression of the MYOD1 gene during the muscular degeneration process in skeletal muscle of the SOD1^{G93A} female mice model of ALS.

FIG. 9. Line showing the change in expression of the NNT gene during the muscular degeneration process in skeletal muscle of the SOD1^{G93A} female mice model of ALS.

FIG. 10. Line showing the change in expression of the SLN gene during the muscular degeneration process in skeletal muscle of the SOD1^{G93A} female mice model of ALS.

FIG. 11. Graph showing the expression level of the Col19α1 gene in human lymphocyte samples of healthy individuals (control) and of ALS patients. Level of expression shown in relation to the expression of the control gene in the samples.

FIG. 12. Graph showing the expression level of the Col19α1 gene in human skeletal muscle samples of healthy individuals (control) and of ALS patients. Level of expression shown in relation to the expression of the control gene in the samples.

FIG. 13. Graph showing the expression level of the IMPA1 gene in human lymphocyte samples of healthy individuals (control) and of ALS patients. Level of expression shown in relation to the expression of the control gene in the samples.

### EXAMPLES

The invention will be illustrated below by some trials carried out by the inventors that demonstrate the specificity and effectiveness of the proposed biomarkers for carrying out the first and second method of the invention for the diagnosis, prognosis and “in vitro” monitoring of muscular degeneration in an individual. These specific examples provided serve to illustrate the nature of the present invention and are included solely for illustrative purposes, so they are not to be interpreted as limitations of the invention which is claimed herein.

### Example 1

### Detection of Prognostic Biomarkers of Muscular Degeneration from Biopsies of the SOD1^{G93A} Transgenic Mouse Model of ALS

### 1.1. Animal Model and the Search for Biomarkers of Muscular Degeneration.

The animal model used was the transgenic mouse of the B6SJL strain that overexpresses the human superoxide dismutase (SOD1) protein mutated in position G93A (SOD1^{G93A}), which is considered as the most suitable model for the study of ALS. Hemizygous animals expressing the mutation were obtained by crossing a male mutant with a healthy female (wild type). Genotyping the progeny was carried out from the DNA extracted from the tail of the animal. The animals were maintained following the general directives for use of laboratory animals. Food and water were supplied ad libitum. Routine microbiological tests did not show evidence of infections with common murine pathogens.

The selection of candidate genes for later testing in muscle biopsies as explained in example 1.2 was mainly based on a prior study of microarrays (Affymetrix) from skeletal muscle samples of 2 month old healthy mice and SOD1^{G93A} transgenic mice and on the results obtained after validation by real-time PCR. From the genes that exhibited differential expression, the following were selected: Ankrd1, Calm1, Col19α1, Fbxo32, Gsr, IMPA1, Mef2c, Mt2, Myf5, Myod1, Myog, Nnt, Pax7, Rrad, Rtn4, also known as NOGO A, Sln and Snx10. Table 1 lists the information corresponding to each gene. In particular, Mef2c, Myf5, Myod1 and Pax7 were included in this study to complete the cascade of myogenic regulatory factors together with Myog, the expression of which was changed in the disease. Similarly, Gsr and NOGO A were included because they showed changes in their expression levels as a consequence of the degenerative process of the disease. Validation by real-time PCR of the change in expression levels of the selected genes was carried out in the StepOne™ Real-Time PCR System (Applied Biosystems) equipment according to the following protocol: incubation at 95° C. for 20 seconds, 40 cycles of 95° C. for 1 second and 60° C. for 20 seconds. The reactions were carried out in a final volume of 5 μL containing a mixture of the reagent 1× TagMan® Fast Universal PCR Master Mix (4352042, No AmpErase® UNG, Applied Biosystems), 1× TagMan® MGB primer and probe and 2 μL of the cDNA diluted 10×. The housekeeping genes that were used for normalisation of the data were 18S rRNA, GAPDH and β-actin.

TABLE 1
Genes selected for subsequent study in muscle
biopsies of the SOD1^{G93A} animal model.

| SYMBOL | GENE ID | FUNCTION |
|---|---|---|
| Ankrd1 | 107765 | Muscular plasticity |
| Calm1 | 12313 | Calcium signal |
| | | modulator |
| | | Motor endplate |
| | | endocytosis mediator |
| Col19α1 | 12823 | Oesophagus muscle |
| | | development and |
| | | differentiation |
| Fbxo32 | 67731 | Promotes muscular |
| | | atrophy |
| | | Reduction of its |
| | | expression levels in |
| | | neuromuscular disorders |
| Gsr | 14782 | Metabolic oxidative |
| | | stress |
| Impa1 | 55980 | Inositol homeostasis |
| | | Target of calbindin |
| Mt2 | 17750 | Properties of binding to |
| | | metals and removing |
| | | free radicals |
| | | Metabolic oxidative |
| | | stress |
| | | Zinc homeostasis |
| Mef2c | 17260 | Maintenance of |
| | | sarcomere integrity |
| | | Muscle differentiation |
| Myod1 | 17927 | Myogenesis |
| | | Muscle differentiation |
| Myf5 | 17877 | Regulator of myogenesis |
| | | Muscle homeostasis |
| Myog | 17928 | Differentiation of muscle |
| | | cells |
| Nnt | 18115 | Glucose homeostasis |
| Pax7 | 18509 | Muscle development |
| Rrad | 56437 | Glucose tolerance and |
| | | insulin sensitivity |
| | | Intracellular regulation of |
| | | calcium signalling |
| NOGO A | 68585 | Inhibitor of axonal |
| | | regeneration |
| Sln | 66402 | Regulator of calcium |
| | | transport |
| | | Muscle contraction- |
| | | relaxation cycles |
| Snx10 | 71982 | Regulation of endosome |
| | | homeostasis |

### 1.2. Extraction of Muscle Biopsies and Search for Prognostic Biomarkers of Muscular Degeneration.

Carrying out muscle biopsies in the animal model of neurodegeneration allows obtaining tissue from the same animal during the disease that can later be analysed in order to find possible prognostic biomarkers because it enables correlating the change of gene expression with the progression of the disease in the animal. However, as the disease advances in these animals, their deterioration is very rapid, so it is necessary that this technique of obtaining biopsies is as little invasive as possible in order to ensure the viability of the animal. For this reason, the area of the gluteus superficialis muscle was chosen in the present invention for obtaining biopsies from SOD1^{G93A} transgenic animals because the manipulation of this area can be carried out in this easily accessible area and does not hinder the mobility of the animal after each intervention. The main advantage of extracting muscle biopsies as carried out in the present invention is that it allows monitoring during the disease in the same animal, keeping it alive. In turn, using this methodology, changes in the expression levels of a biomarker can be followed more rigorously and more closely to the real development of the disease in a tissue that is seriously damaged as a consequence of the disease, in this case the skeletal muscle. The extraction process was divided into various phases:

1. Pre-Medication and Preparation of the Animal:

10-20 minutes before obtaining the biopsy, the analgesic Meloxicam 2 mg/kg (Metacam©) was administered subcutaneously and the area of the gluteus superficialis muscle was shaved (approximately 4 cm²). After shaving, the area was disinfected with 70° alcohol and iodinated povidone.

2. Surgery:

The animal was anaesthetised with isoflurane in an induction chamber (4-5% of isoflurane), followed by fitting the animal with a mask and reducing the flow to 1.5-2%. When the animal was in the mask, absence of reflexes was checked and a humidifying gel was applied over the eyes of the animal to prevent any damage to the cornea during and after the procedure (Lubrithal©). An incision <1 cm was made by scalpel in the skin at the level of the gluteus superficialis muscle, the connective tissue was withdrawn to access the muscle tissue and a small hole of muscle of approximately 1 mm² was cut. Once the biopsy was extracted, the skin was closed by a staple (EZ 9 mm clip) and a healing ointment (Aloe vet©) applied to facilitate the closure of the wound in a short time. Finally, it was rehydrated with 0.9% physiological saline. After stopping the flow of anaesthetic, the animal was checked for reflexes and was returned to its corresponding tray.

3. Post-Surgery:

during this process, a warm environment was maintained using an IR lamp to facilitate the animal's recovery. Similarly, a small amount of hydrated food and paper were added to facilitate recovery during the first few hours after the extraction of the biopsy. During the first 24 hours, the animals were checked 2 times and a week after surgery the staples were removed.

This extraction process was carried out on 48 transgenic animals (24 females and 24 males) so that two biopsies were extracted from the hind limbs, alternating the limbs, at 75 days (early stage of the disease) and at 105 days (advanced stage of the disease) (Miana-Mena, et al., 2005. Amyotroph Lateral Scler Other Motor Neuron Disord., 6(1):55-62) and finally a third biopsy prior to sacrifice of the animal (terminal stage of the disease). The animal was sacrificed when, placed supine on a tray it was not capable of righting itself within 30 seconds. Each biopsy was kept in an Eppendorf tube with RNAlater (Ambion) in order to preserve the tissue and avoid the degradation of the RNA.

After all the biopsies had been performed, the extracted samples were processed to obtain in a first pass the total RNA (Micro Kit Protocol, Quiagen, that enables obtaining RNA from very small samples in optimum condition) and finally the corresponding complementary DNA (SuperScript™ First-Strand Synthesis System kit, Invitrogen).

The expression of the selected genes as explained in example 1.1 (Ankrd1, Calm1, Col19α1, Fbxo32, Gsr, IMPA1, Mef2c, Mt2, Myf5, Myod1, Myog, Nnt, Pax7, Rrad, Rtn4, also known as NOGO A, Sln and Snx10) were studied in the muscle biopsies to determine if they could be considered prognostic markers of muscular degeneration.

Validation by real-time PCR of the change in expression levels of the selected genes was carried out in the StepOne™ Real-Time PCR System (Applied Biosystems) equipment according to the following protocol: incubation at 95° C. for 20 seconds, 40 cycles of 95° C. for 1 second and 60° C. for 20 seconds. The reactions were carried out in a final volume of 5 μL containing a mixture of 1× TaqMan® Fast Universal PCR Master Mix (4352042, No AmpErase® UNG, Applied Biosystems) reagent, 1× TaqMan® MGB primer and probe and 2 μL of the cDNA diluted 10×. The housekeeping genes that were used for the normalisation of the data were 18S rRNA, GAPDH and β-actin, the expression of which are maintained constant over the duration of the disease.

Of the 17 selected genes, those whose gene expression over the duration of the disease was correlated with its progression could be identified as prognostic markers of muscular degeneration. It is important to highlight that owing to the differences in the behaviour of the skeletal muscle between the sexes in a situation of degeneration, the data from males and females were analysed separately.

In the study of correlation with the progression of the disease, the ΔCt values obtained from each biopsied sample from SOD1^{G93A} transgenic male and female animals at 75, 105 days and at the time of sacrifice of each animal were analysed. In this way, for each animal, male or female, and for each gene, 3 threshold values of the cycle (Ct) obtained in the corresponding real-time PCR and normalised (ΔCt) were obtained for the housekeeping genes, corresponding to the three ages under study. These values were represented graphically to calculate the slope of the line obtained for each gene. In this way, for each gene and in each sex, a group of slopes was obtained, each value belonging to one animal, that was subjected to statistical analysis as explained below.

The results of the slopes obtained were treated with SPSS 15.0 software to determine Pearson's linear correlation coefficient, R. This coefficient estimates the degree of linearity of the slopes with respect to the age of sacrifice in each case, which varied in the range −1 to 1, so that when the coefficient reached the value 1, the variables under study showed perfect linear correlation. All the values were expressed as the mean±standard deviation. Statistical significance was reached with p<0.05.

### 1.3. Results.

The data obtained in the study of correlation with progression of muscular degeneration showed that of the 17 genes validated by real-time PCR, only 2 were linearly correlated with progression in both sexes (Col19α1 and NOGO A). Pearson's coefficient, R, for both genes was positive in both males (Table 2) and females (Table 3), indicating that the two genes gradually reduced their ΔCt value in both sexes as the degenerative process advanced and, therefore, samples of skeletal muscle from individuals in an earlier stage of the muscular degeneration process showed higher ΔCt values of either of these two genes compared to samples coming from individuals in more advanced degenerative stages. NOGO A induces instability in the neuromuscular joint when overexpressed in muscle, which is consistent with the fact observed here that the higher the NOGO A expression, the more advanced was the state of muscular degeneration (taking into account that ΔCt is inversely proportional to the relative concentration of the gene).

TABLE 2
Slopes calculated in males from the ΔCt values for
each gene and the corresponding Pearson's coefficients
(*p < 0.05, **p < 0.01). Col19α1 and NOGO
A were the only genes in which a correlation with the
progression of muscular degeneration was found.

| | MALES | | |
|---|---|---|---|
| | SLOPE | PEARSON'S | STATISTICAL |
| GENE | (MEAN ± SD) | COEFFICIENT | SIGNIFICANCE |
| Ankrd1 | −0.1159 ± 0.067 | 0.409 | p = 0.212 |
| Calm1 | −0.0101 ± 0.021 | −0.516 | p = 0.104 |
| Col19α1 | −0.0803 ± 0.087 | 0.664* | p = 0.026 |
| Fbxo32 | −0.036 ± 0.033 | 0.383 | p = 0.245 |
| Gsr | −0.024 ± 0.011 | 0.347 | p = 0.296 |
| Impa1 | −0.0144 ± 0.017 | 0.508 | p = 0.110 |
| Mt2 | −0.0016 ± 0.032 | 0.564 | p = 0.07 |
| Mef2c | −0.0206 ± 0.021 | −0.074 | p = 0.828 |
| Myod1 | −0.039 ± 0.038 | −0.087 | p = 0.800 |
| Myf5 | −0.0425 ± 0.030 | 0.157 | p = 0.645 |
| Myog | −0.0732 ± 0.044 | 0.488 | p = 0.127 |
| Nnt | −0.0035 ± 0.019 | 0.207 | p = 0.542 |
| Pax7 | −0.0028 ± 0.024 | −0.299 | p = 0.372 |
| Rrad | −0.0883 ± 0.062 | 0.357 | p = 0.282 |
| Rtn4 | −0.0034 ± 0.007 | 0.755** | p = 0.007 |
| Sln | −0.1481 ± 0.058 | 0.544 | p = 0.084 |
| Snx10 | −0.0226 ± 0.018 | 0.307 | p = 0.359 |

These two genes were the only genes in male transgenic animals where a significant linear correlation between the change in their expression levels and the progression of muscular degeneration was found. In female transgenic animals a degree of significant positive correlation with the progression of degeneration was found in another six genes (Ankrd1, Myod1, Myog, Nnt, Sin and Snx10) (Table 3). These genes gradually reduced their ΔCt value in females as the degeneration process progressed and, therefore, samples of skeletal muscle from female individuals in an early stage of the muscular degeneration process had higher values of ΔCt in any of these six genes in comparison with samples from female individuals in more advanced degenerative states. This result suggests that as the neurodegenerative process advances, the increase in the expression of any of these six genes indicates a worsening of the degenerative process because processes such as differentiation, tissue integrity and glucose and calcium homeostasis are changed.

TABLE 3
Slopes calculated in females from the ΔCt values of each
gene and the corresponding Pearson's coefficients (*p <
0.05, **p < 0.01). Of the 17 genes tested, correlations
with the progression of the disease was identified in 8 genes
(Ankrd1, Col19α1, Myod1, Myog, Nnt, NOGO A, Sln and Snx10).

| | FEMALES | | |
|---|---|---|---|
| | SLOPE | PEARSON'S | STATISTICAL |
| GENE | (MEAN ± SD) | COEFFICIENT | SIGNIFICANCE |
| Ankrd1 | −0.1213 ± 0.063 | 0.781** | p = 0.033 |
| Calm1 | −0.0096 ± 0.028 | 0.345 | p = 0.266 |
| Col19α1 | −0.1160 ± 0.046 | 0.862** | p = 0.0003 |
| Fbxo32 | −0.0221 ± 0.034 | 0.487 | p = 0.108 |
| Gsr | −0.0148 ± 0.033 | 0.386 | p = 0.215 |
| Impa1 | −0.0109 ± 0.021 | 0.253 | p = 0.427 |
| Mt2 | −0.0121 ± 0.051 | 0.495 | p = 0.102 |
| Mef2c | 0.0436 ± 0.025 | 0.502 | p = 0.097 |
| Myod1 | −0.0589 ± 0.029 | 0.692* | p = 0.013 |
| Myf5 | −0.0481 ± 0.032 | 0.495 | p = 0.101 |
| Myog | −0.0721 ± 0.043 | 0.745** | p = 0.005 |
| Nnt | −0.0214 ± 0.056 | 0.605* | p = 0.037 |
| Pax7 | −0.027 ± 0.022 | 0.379 | p = 0.224 |
| Rrad | −0.0742 ± 0.042 | 0.468 | p = 0.125 |
| Rtn4 | −0.0141 ± 0.015 | 0.7815* | p = 0.033 |
| Sln | −0.1215 ± 0.041 | 0.773** | p = 0.033 |
| Snx10 | 0.0267 ± 0.027 | 0.654* | p = 0.021 |

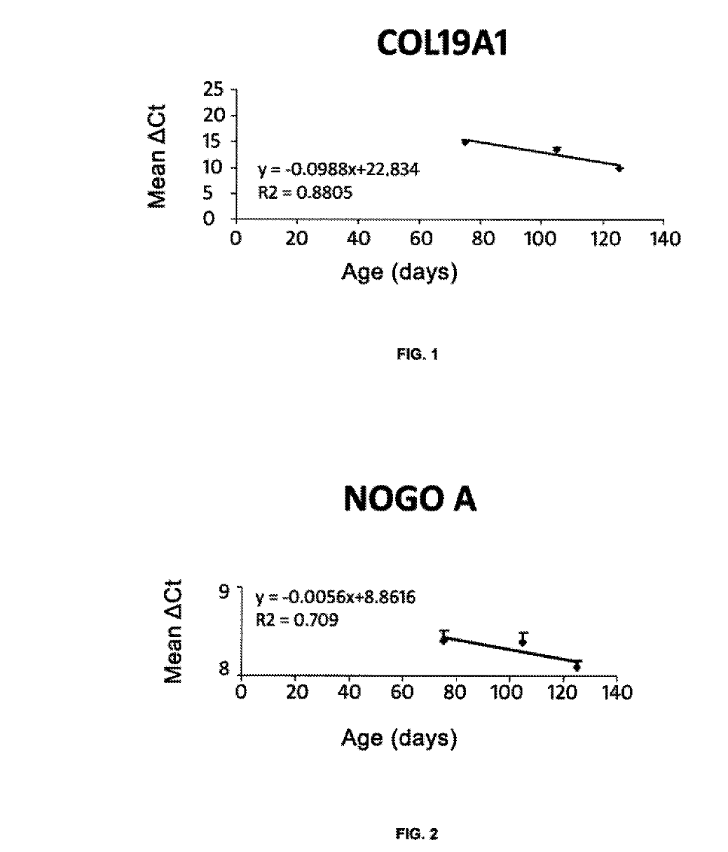

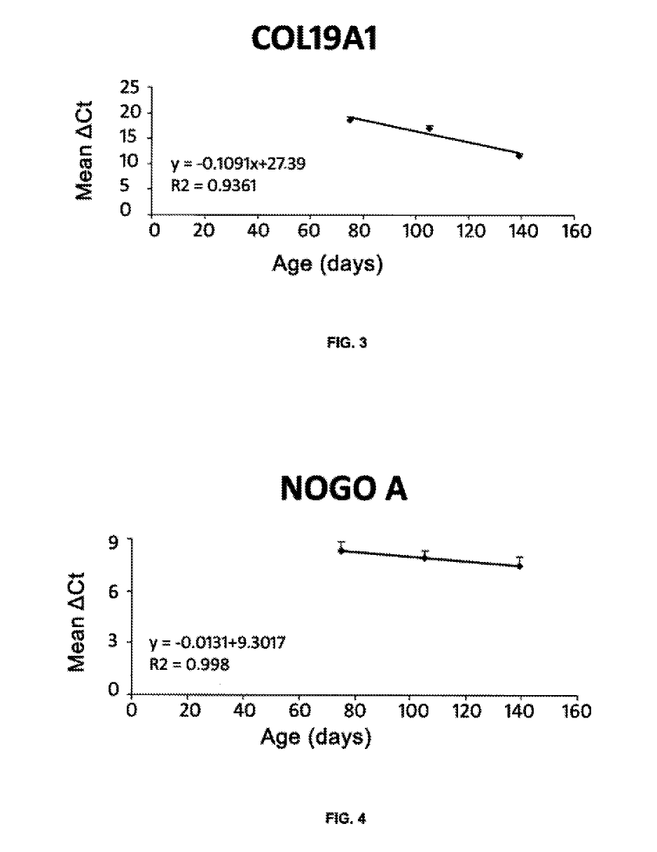

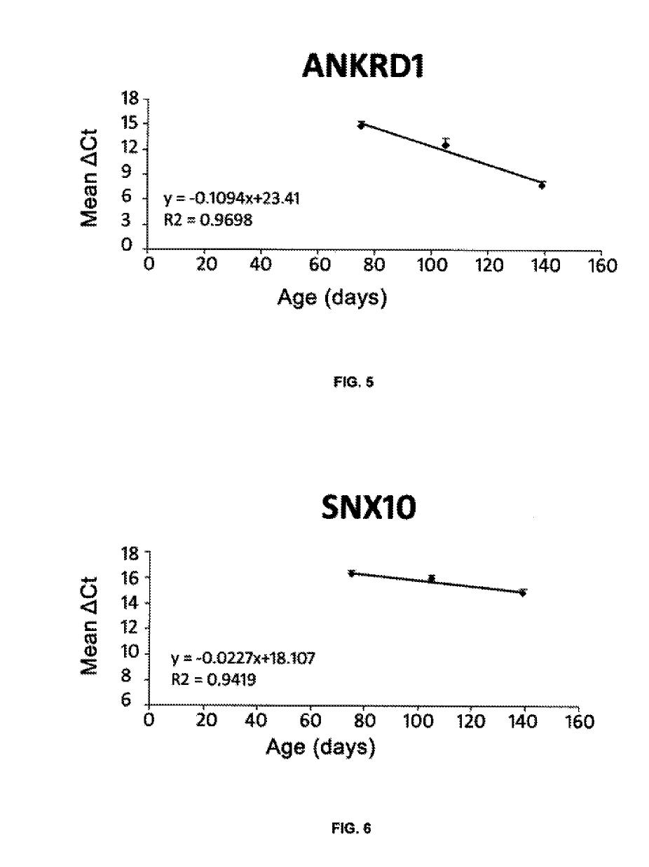

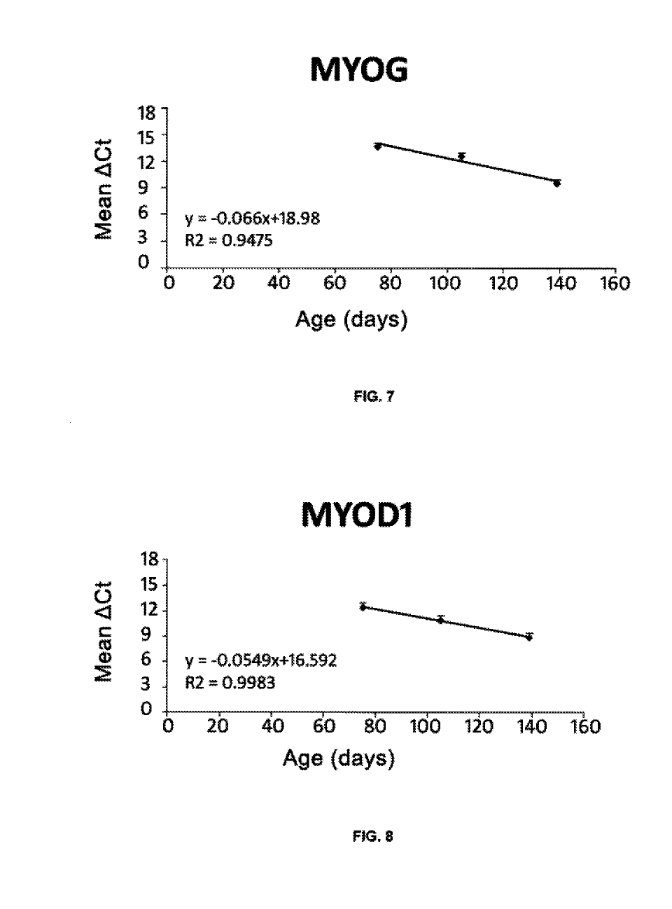

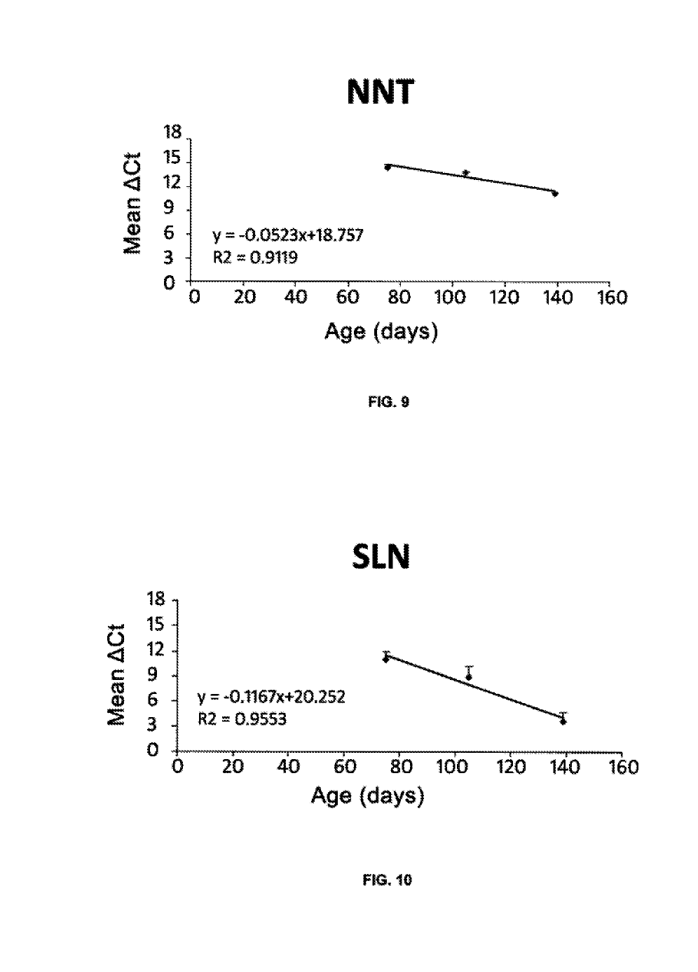

The mean ΔCt corresponding to each gene in each sex and for each of the three disease states were calculated from the ΔCt values of the individuals. These mean values were represented graphically (FIGS. 1-10) and the slopes of the lines obtained were calculated, so that these lines and slopes represented what could be considered as the normal evolution of the change in gene expression of each of the genes proposed as prognostic biomarkers of the degenerative process. Therefore, the values of these slopes can be called “reference values”.

Thus, to carry out the study of an individual with muscular degeneration, at least two isolated biological samples of skeletal muscle should be taken from the patient and the ΔCt values of the genes proposed here as prognostic biomarkers should be determined in order to represent the values obtained on a line, from which the slope can be obtained. Any significant deviation of the slope thus obtained compared to the reference slope (which would be that obtained for each gene according to the previously explained calculation) would be indicative of a change in the speed of progression of muscular degeneration.

Table 4 shows that all the reference values are negative, given that the ΔCt values of the proposed biomarkers of the invention reduced during the degenerative process.

TABLE 4
Control values or reference amounts for each gene proposed as
prognostic biomarker of muscular degeneration in each sex.

| SLOPES | FEMALES | MALES |
|---|---|---|
| ANKRD1 | −0.1094 | — |
| SNX10 | −0.0227 | — |
| MYOG | −0.066 | — |
| MYOD1 | −0.0549 | — |
| NNT | −0.0523 | — |
| SLN | −0.1167 | — |
| COL19A1 | −0.1091 | −0.0988 |
| NOGO A | −0.0131 | −0.0056 |

### Example 2

### Detection of Diagnostic Biomarkers of Muscular Degeneration from Human Biological Samples

2.1. Samples from Patients.

Samples were obtained from patients and controls after obtaining informed consent. One sample was taken from each patient.

Lymphocytes: from 10 ml of total blood, the subpopulation of lymphocytes was isolated in a Ficoll gradient (Ficoll-Paque™ Plus; GE Healthcare) and total RNA was extracted with TriReagent (Sigma-Aldrich Co.). The amount and purity of the extracted RNA was determined in a NanoDrop spectrophotometer and its integrity was checked by viewing the bands corresponding to the 285 and 18S rRNA in agarose gel electrophoresis. Complementary DNA was obtained from 1 μg RNA (High Capacity cDNA RT kit; Applied Biosystems). Samples were taken at the time of definitive diagnosis of the disease.

Muscle: muscle biopsies were obtained from the biceps brachii by open biopsy after administering subcutaneous local anaesthesia. Immediately after extraction, the tissue was frozen in liquid nitrogen. For isolation of the RNA and subsequent complementary DNA synthesis, 30-40 mg of tissue was taken, and the procedure was the same as with the lymphocytes. The time of sample collection in this case was variable.

### 2.2 Analysis of Gene Expression

Gene expression was analysed by real-time PCR from the complementary DNA obtained. In lymphocytes, studies were carried out on the Col19α1 gene and the IMPA1 gene and the samples analysed were 47 controls with an age range of 59.9±8.69 years, of which 29 were men and 18 women, and 53 sporadic ALS patients with an age range of 59.1±15.15 years, with a distribution of 30 men and 23 women. In muscle, the study was repeated of Col19α1 gene expression and the analysed samples were 4 controls with an age range of 77±6.4 years, of which 4 were women and 8 sporadic ALS patients with an age range of 58.26±5.59 years, with a distribution of 7 men and 1 woman.

The PCR reaction was carried out in a 7500 Real-Time PCR System (Applied Biosystems) equipment with inventoried TaqMan probes (Applied Biosystems), the efficiency of which had been tested and in all cases was close to 100%. All the reactions were carried out in triplicate and the expression of GAPDH as an endogenous gene was used for normalisation. The data were analysed quantitatively against those of a calibrating sample by the ΔΔCt method. Briefly, for each sample, the Ct value of the normalising gene (GAPDH) was subtracted from the Ct value of the analysed gene or target (ΔCt=Ct_{target}−Ct_{endogenous or target}); from the ΔCt thus obtained the normalised value (ΔCt) of the calibrator (ΔΔCt) was subtracted and the values were linearized with the calculation 2^{-(ΔΔCt)}, so that the expression of the target gene of each sample is shown in relation to the expression of this same gene in the calibrating sample. The calibrating sample was a sample from a healthy control.

Statistical analysis was carried out by the SPSS statistical program. Non parametric tests were used in all cases to ensure that the possibly non-normal distribution of the various samples did not interfere in the results. Variables were compared using the Wilcoxon test.

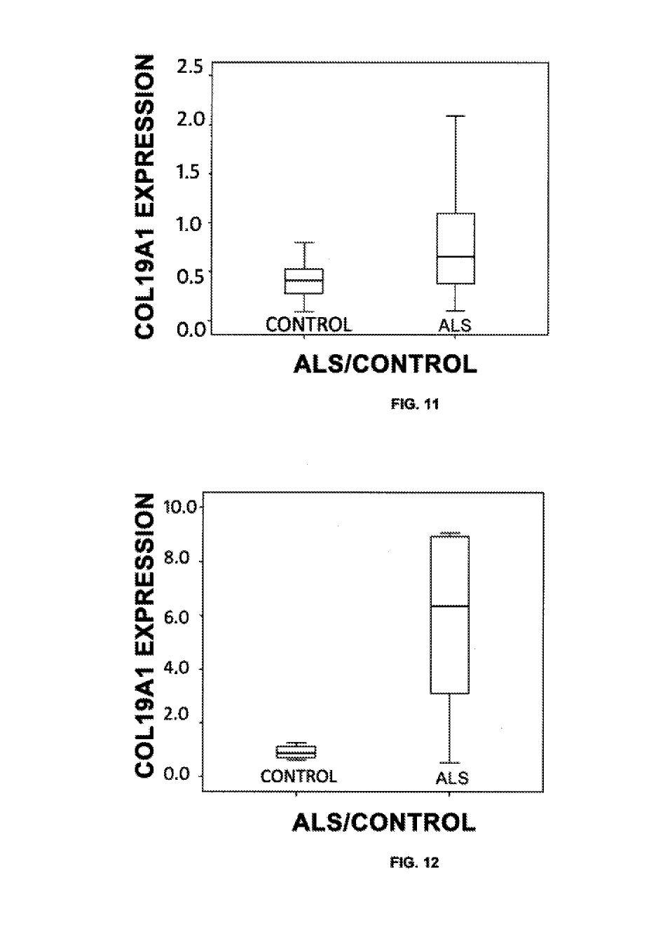

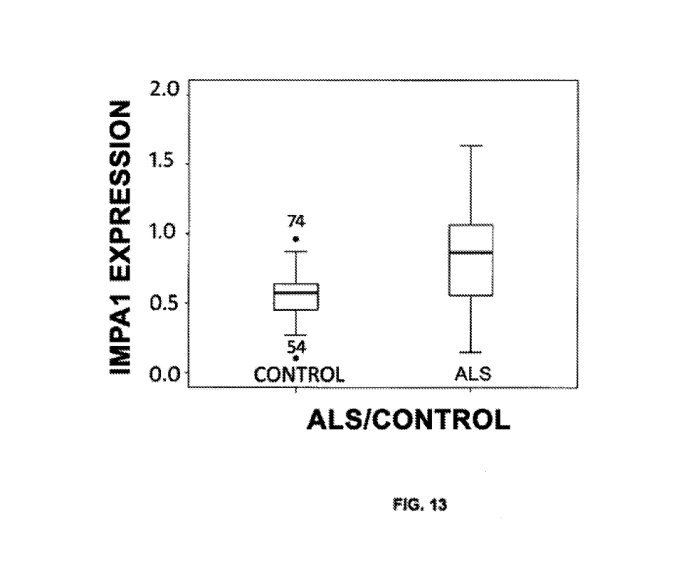

The results of the Col19α1 and IMPA1 genes in human lymphocyte samples were statistically significant between the ALS patient group and the control group. These results are shown in FIG. 11, for Col19α1, and FIG. 13, for IMPA1. The mean of the Col19α1 gene expression in the control group was 0.46±0.25 (expression relative to the expression of the control gene) (0.10 to 1.21) and the mean expression of this gene in the ALS group was 0.80±0.52 (0.10 to 2.09), and this difference was statistically significant (Wilcoxon test): 0.0166. The mean of the IMPA1 gene expression in the control group was 0.61±0.25 (expression relative to the expression of the control gene) (0.10 to 1.35) and the mean expression of this gene in the ALS group was 0.84±0.35 (0.15 to 1.64), and this difference was statistically significant (Wilcoxon test): 0.0028.

The results of the Col19α1 gene expression in muscle biopsies of patients with ALS compared to controls showed a more marked change of this gene than in lymphocytes, as shown in FIG. 12, and the difference was statistically significant: 0.0047.

1. (canceled)

2. (canceled)

3. (canceled)

4. (canceled)

5. (canceled)

6. (canceled)

7. (canceled)

8. Method for the “in vitro” diagnosis of muscular degeneration in an individual comprising:
a. determining the amount of Col19α1 gene expression product in an isolated biological sample from an individual, and
b. comparing the amount determined in step (a) with a reference amount, wherein the reference amount is the amount of Col19α1 gene expression product in an isolated biological sample of an individual that does not exhibit muscular degeneration.

9. (canceled)

10. Method according to claim 8 wherein the isolated biological sample is a lymphocyte or skeletal muscle.

11. Method according to claim 8 that additionally comprises:
c. assigning the individual of step (a) to the group of patients with muscular degeneration when the amount determined in step (a) is significantly higher than the reference amount.

12. Method according to claim 8 wherein the isolated biological sample is a lymphocyte, further comprising:
d. determining the amount of IMPA1 gene expression product in the lymphocyte of the individual of step (a), and
e. comparing the amount determined in step (d) with a reference amount.

13. Method according to claim 12 further comprising:
f. assigning the individual of step (a) to the group of patients with muscular degeneration when the amount determined in steps (a) and (d) are significantly higher than the reference amount, wherein the reference amount is the amount of IMPA1 gene expression product in a lymphocyte of an individual that does not exhibit muscular degeneration.

14. (canceled)

15. Method for the “in vitro” prognosis and monitoring of muscular degeneration in an individual comprising:
a. determining the ΔCt value of the Col19α1 gene in an isolated biological sample of skeletal muscle of an individual that exhibits muscular degeneration,
b. determining the ΔCt value of the Col19α1 gene in a second isolated biological sample of skeletal muscle of the individual of step (a) obtained at least 1 month after obtaining the isolated biological sample of step (a),
c. calculating the slope of a line obtained after connecting the values determined in steps (a) and (b), and
d. comparing the slope calculated in step (c) to a reference value.

16. Method of claim 15 wherein the reference value of step (d) is the slope of a line obtained after connecting the mean of the ΔCt values of the Col19α1 gene in various isolated biological samples of skeletal muscle of various individuals exhibiting muscular degeneration to the mean of the ΔCt values of the Col19α1 gene in various isolated biological samples of skeletal muscle of various individuals exhibiting muscular degeneration obtained at least 1 month after obtaining the first biological sample.

17. Method according to claim 15 further comprising:
e. assigning the individual of step (a) to the group of patients with high speed of progression of muscular degeneration when the slope calculated in step (c) is significantly less than the reference value.

18. Method according to claim 15 further comprising:
f. determining the ΔCt value of the NOGO A gene in the isolated biological sample of step (a),
g. determining the ΔCt value of the NOGO A gene in the isolated biological sample of step (b),
h. calculating the slope of a line obtained after connecting the values determined in steps (f) and (g), and
i. comparing the slope calculated in step (h) to a reference value.

19. Method according to claim 18 wherein the reference value of step (i) is the slope of a line obtained after joining the mean of the ΔCt values of the NOGO A gene in various isolated biological samples of skeletal muscle of various individuals exhibiting muscular degeneration to the mean of the ΔCt values of the NOGO A gene in various isolated biological samples of skeletal muscle of various individuals exhibiting muscular degeneration obtained at least 1 month after obtaining the first biological sample.

20. Method according to claim 18 further comprising:
j. assigning the individual of step (a) to the group of patients with high speed of progression of muscular degeneration when the slopes calculated in steps (c) and (h) are significantly lower than the reference values.

21. Method according to claim 18 wherein the individual is a female and additionally comprising:
k. determining the ΔCt value of at least one gene selected from the list comprising: ANKRD1, SNX10, MYOG, MYOD1, NNT and SLN in the isolated biological sample of step (a),
l. determining the ΔCt value of the gene(s) selected in step (k) in the isolated biological sample of step (b),
m. calculating the slope(s) of the line(s) obtained for each gene after connecting the values determined in steps (k) and (l), and
n. comparing the slope(s) calculated in step (m) to a reference value.

22. Method according to claim 21 further comprising:
o. assigning the individual of step (a) to the group of patients with high speed of progression of muscular degeneration when the slopes calculated in steps (c), (h) and (m) are significantly lower than the reference value.

23. Method according to claim 21 wherein the reference value of step (n) is the slope of a line obtained after connecting the mean of the ΔCt values of the ANKRD1, SNX10, MYOG, MYOD1, NNT or SLN gene in various isolated biological samples of skeletal muscle of various female individuals exhibiting muscular degeneration to the mean of the ΔCt values of the ANKRD1, SNX10, MYOG, MYOD1, NNT or SLN gene in various isolated biological samples of skeletal muscle of various female individuals exhibiting muscular degeneration obtained at least 1 month after taking the first biological sample.

24. Method according to any of the claim 15 wherein the muscular degeneration is caused by a motor neurone disease.

25. Method according to claim 24 wherein the motor neurone disease is selected from the list consisting of: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy and amyotrophic lateral sclerosis.

26. (canceled)

27. (canceled)

28. (canceled)

29. Kit comprising specific primers, probes or antibodies, or any of their combinations, for the Col19α1 gene.

30. Kit according to claim 29 further comprising specific primers, probes or antibodies, or any of their combinations, for the NOGO A gene and/or for the IMPA1 gene and/or comprising specific primers, probes or antibodies, or any of their combinations, for at least one of the genes selected from the list consisting of: ANKRD1, SNX10, MYOG, MYOD1, NNT and SLN.

31. (canceled)

32. (canceled)

33. (canceled)

34. (canceled)

35. (canceled)

36. Method according to claim 8 wherein the muscular degeneration is caused by a motor neurone disease.

37. Method according to claim 36 wherein the motor neurone disease is selected from the list consisting of: spinal muscular atrophy, bulbo-spinal atrophy, progressive muscular atrophy, primary lateral sclerosis, hereditary spastic paraplegia, tropical spastic paraplegia, bulbar palsy, pseudobulbar palsy, adrenomyeloneuropathy, lathyrism, acute poliomyelitis, post-polio syndrome, multifocal motor apnoea, benign focal amyotrophy and amyotrophic lateral sclerosis.
